(19) United States
(12) Patent Application Publication
(10) Pub. No.: US 2024/0207575 A1
(43) Pub. Date: Jun. 27, 2024

(54) METHOD TO IMPROVE AND MINIATURIZE A MAGNETIC POSITION SENSOR FOR A CATHETER OR TOOL

(71) Applicant: Covidien LP, Mansfield, MA (US)

(72) Inventors: Nathan J. Knutson, Long Lake, MN (US); Franklin J. Burquest, Crystal, MN (US); Evan M. Gustafson, Sioux Falls, SD (US); Matthew A. Rootes, Crystal, MN (US); Bethany A. Palmer, Coon Rapids, MN (US); Tina M. Berthiaume, Champlin, MN (US)

(73) Assignees: Covidien LP, Mansfield, MA (US); Covidien LP, Mansfield, MA (US)

(21) Appl. No.: 18/288,949

(22) PCT Filed: May 27, 2022
(86) PCT No.: PCT/US22/31378

§ 371 Date: Oct. 30, 2023

Related U.S. Application Data
(60) Provisional application No. 63/194,706, filed on May 28, 2021.

(51) Int. Cl. A61M 25/01; A61M 25/00

(52) U.S. Cl. CPC A61M 25/0127; A61M 25/0045; A61M 25/007; A61M 25/0012; A61M 2025/015

(57) ABSTRACT

A catheter including a catheter body (60) formed of a polymeric material and having a lumen therethrough, a metallic reinforcement structure in contact with the polymeric material, a core, and a sensor wire with a first portion (71) formed in a plurality of windings wrapped around the core and a second portion formed as a twisted pair (80) extending the catheter body (60). The catheter may include a pull wire anchored to the core.

### FIELD

The present disclosure relates to elongated catheters and, more specifically, to methods of manufacturing elongated catheters having a sensor and a working channel therethrough.

### BACKGROUND

A common interventional procedure in the field of pulmonary medicine is bronchoscopy, in which a bronchoscope is inserted into the airways through the patient's nose or mouth. The structure of a bronchoscope generally includes a long, thin, flexible tube that typically contains three elements: an illumination assembly for illuminating the region distal to the bronchoscope's tip via an optical fiber connected to an external light source; an imaging assembly for delivering back a video image from the bronchoscope's distal tip; and a lumen or working channel through which instruments may be inserted, including, but not limited to, placement instruments (e.g., guide wires), diagnostic instruments (e.g., biopsy tools) and therapeutic instruments (e.g., treatment catheters or laser, cryogenic, radio frequency, or microwave tissue treatment probes).

During some procedures (e.g., microwave ablation and biopsy), a catheter having an extended working channel may be inserted through a working channel of the bronchoscope to enable navigation to sites that are typically too remote, or have luminal diameters too small, for the bronchoscope. The catheter may have a locatable sensor at its distal end to assist in guiding the catheter to targeted tissue. When the distal end of the catheter is positioned adjacent targeted tissue, an instrument may be inserted through the working channel of the catheter to perform a procedure on the targeted tissue (e.g., perform a biopsy or ablation of the targeted tissue).

While current methods of forming the sensor create a functional device, improvements that can improve the signal and are more easily manufactured are always desired.

### SUMMARY

One aspect of the disclosure is directed to a catheter including: an inner liner defining a lumen, an outer liner formed over the inner liner, a metallic reinforcement structure between the inner liner and the outer liner, a first sleeve operably connected to the outer liner. The catheter also includes a core in communication with the inner liner and having a lumen formed therein of substantially the same diameter as the lumen in the inner liner and a reduced diameter portion; a pull wire traversing the first sleeve and anchored to the core. The catheter also includes a wire, having a first portion of the wire is formed in a plurality of windings wrapped around the reduced diameter portion of the core and a second portion of the wire formed as a twisted pair extending proximally along the outer liner. The catheter also includes an outer sheath exterior to the outer liner, the first sleeve, and the core.

Implementations of this aspect of the disclosure may include one or more of the following features. The catheter further including a second sleeve operably connected to the outer liner and within the outer sheath, where the twisted pair extends through the second sleeve. The catheter further including a plurality of fenestrations formed in the outer liner and configured to allow the pull wire to articulate a distal portion of the catheter. The catheter where twisted pair wraps around the outer liner and is captured by the outer liner. The catheter where the core includes a channel formed therein for passage of the twisted pair. The catheter where the core includes a cylinder formed on a proximal portion, the cylinder having an inner diameter substantially the same as the lumen in the inner liner. The catheter where the plurality of windings are formed on the cylinder.

One aspect of the disclosure is directed to a catheter assembly including: a catheter body formed of one or more polymeric materials, the catheter body defining a lumen therethrough; a metallic reinforcement structure in contact with the one or more polymeric materials; a core distal of the metallic reinforcement structure and the one or more polymeric materials; and a wire, where a first portion of the wire is formed in a plurality of windings wrapped around the core and a second portion of the wire formed as a twisted pair extending proximally along the catheter body.

Implementations of this aspect of the disclosure may include one or more of the following features. The catheter assembly where the core includes a lumen formed therein substantially the same diameter as the lumen in the catheter body. The catheter assembly where the core includes a channel formed therein for passage of the twisted pair. The catheter assembly where the core includes a reduced diameter portion for receiving the wire and forming the plurality of windings. The catheter assembly further including a sleeve formed in the catheter body. The catheter assembly further including a pull wire traversing the sleeve and attached to the core, where actuation of the pull wire articulates a distal portion of the catheter assembly. The catheter assembly further including a plurality of fenestrations formed in the catheter body and configured to allow the pull wire to articulate a distal portion of the catheter. The catheter where the catheter body includes an outer liner and the twisted pair is captured by the outer liner. The catheter where the core includes a channel formed therein for passage of the twisted pair. The catheter where the catheter body includes an inner liner and the core includes a cylinder formed on a proximal portion, the cylinder having an inner diameter substantially the same as a diameter of a lumen in the inner liner. The catheter where the plurality of windings are formed on the cylinder.

One aspect of the disclosure is directed to a catheter assembly including: a catheter body formed of one or more polymeric materials, the catheter body defining a lumen therethrough; a metallic reinforcement structure in contact with the one or more polymeric materials; a core distal of the metallic reinforcement structure and embedded in a wall of the catheter body, the core covered by at least one of the one or more polymeric materials, the core extending partially around a diameter of the catheter body; and a wire, where a first portion of the wire is formed in a plurality of windings wrapped around the core and a second portion of the wire formed as a twisted pair extending proximally along the catheter body.

Implementations of this aspect of the disclosure may include one or more of the following features. The catheter assembly further including a sleeve operably connected to the catheter body and embedded in the one or more polymeric materials, where the twisted pair traverses the sleeve.

### BRIEF DESCRIPTION OF THE DRAWINGS

Various exemplary embodiments are illustrated in the accompanying figures. It will be appreciated that for simplicity and clarity of the illustration, elements shown in the figures referenced below are not necessarily drawn to scale. Also, where considered appropriate, reference numerals may be repeated among the figures to indicate like, corresponding or analogous elements.

FIG. 1 is a profile view of a catheter in accordance with aspects of the disclosure;

FIG. 2 is a cross sectional view of the catheter of FIG. 1 in accordance with aspects of the disclosure;

FIG. 3 is a cross sectional view of the catheter of FIG. 1 proximate a sensor in accordance with aspects of the disclosure;

FIG. 4 is a profile view of a distal portion of a sensor in accordance with aspects of the disclosure;

FIG. 5 is a perspective view of a core for forming a sensor in accordance with aspects of the disclosure;

FIG. 6A is a perspective view of a further catheter in accordance with the disclosure;

FIG. 6B is a cross-sectional view of the distal portion of the catheter of FIG. 6A;

FIG. 7 is a cross-section view of a distal portion of a catheter in accordance with the disclosure.

FIG. 8A is a perspective view of a distal portion of a catheter in accordance with a further aspect of the disclosure;

FIG. 8B is a perspective view of a distal portion of a catheter in accordance with a further aspect of the disclosure; and

FIG. 8C is an end view of a distal portion of a catheter in accordance with a further aspect of the disclosure.

### DETAILED DESCRIPTION

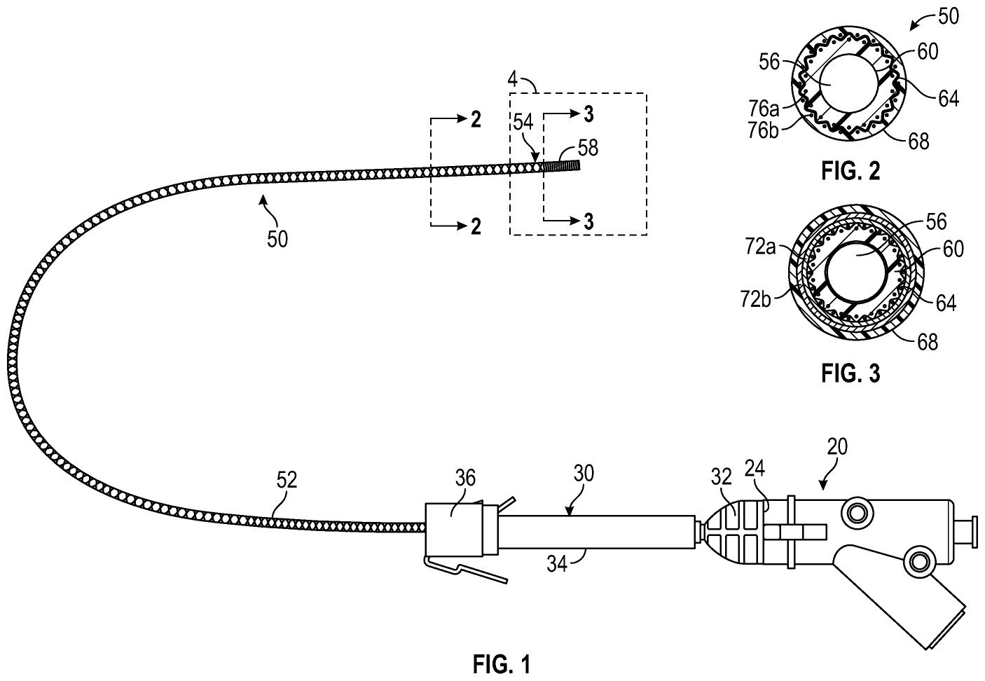

This disclosure is directed methods of miniaturizing electromagnetic sensors as might be incorporated on a catheter or surgical tool. Referring now to FIG. 1, a catheter 10 is provided in accordance with the disclosure and includes a handle assembly 20, a telescopic channel 30, and an elongated catheter body 50 having a proximal end portion 52 and a distal end portion 54. The handle assembly 20 is coupled to the proximal end portion 52 of the catheter body 50 to permit a clinician to manipulate the catheter assembly 10.

The telescopic channel 30 is positioned between the handle assembly 20 and the proximal end portion 52 of the catheter body 50 to provide a means of advancing the catheter body 50, for example through a working channel of a bronchoscope to which the catheter assembly is attached. The telescopic channel 30 includes a proximal or first end portion 32 that is coupled to a distal end portion 24 of the handle assembly 20 and a distal or second end portion 36 that is configured to couple the catheter assembly 10 to a bronchoscope (not shown). The telescopic channel 30 includes an extendable body portion 34 between the first and second end portions 32, 36 that is expandable along a longitudinal axis and substantially rigid transverse to the longitudinal axis. The extendable body portion 34 allows the first end portion 32 to translate along and rotate about the longitudinal axis relative to the second end portion 36. When the first end portion 32 is coupled to the handle assembly 20, the proximal end portion 52 of the catheter body 50 translates and rotates with the first end portion 32 of the telescopic channel 30 by manipulation of the handle 20.

With additional reference to FIGS. 2 and 3, the catheter body 50 defines a working channel 56 along a length thereof. The working channel 56 allows instruments (not shown) to be inserted through the catheter body 50 to biopsy or treat targeted tissue adjacent the distal end portion 54 of the catheter body 50. The catheter body 50 includes an inner liner 60, a metallic reinforcement structure 64, and an outer coating 68. The inner liner 60 defines the working channel 56 that passes entirely through the catheter body 50. It is contemplated that the catheter body 50 may be constructed without the inner liner 60 such that the metallic reinforcement structure 64 defines the working channel 56. In some embodiments the metallic reinforcement structure 64 may be a braid formed of steel such as stainless steel or nitinol. Further the metallic reinforcement structure 64 may be a laser cut structure such as a tube or a welded wire structure in addition to a braid.

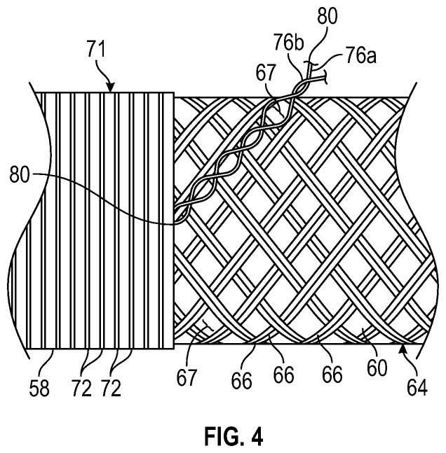

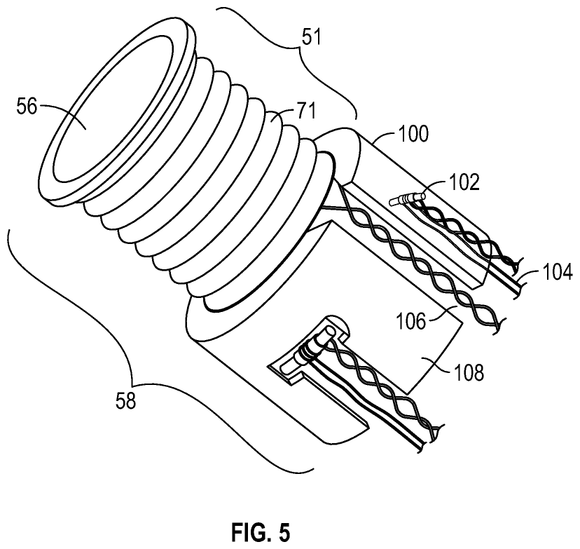

With additional reference to FIG. 4, a sensor 58 is formed of a continuous wire 71 wrapped over the metallic reinforcement structure 64 and covered by the outer coating 68 to form the sensor 58. The wire 71 includes leads 76a, 76b that are twisted together to form a twisted pair 80 that is coiled about the metallic reinforcement structure 64 along the proximal end portion 52 of the catheter body 50.

The inner liner 60 and the outer coating 68 are formed from polymer tubes, as detailed below, which may be made from of a reflowable polymer material (e.g., thermoplastic polymers or polytetrafluoroethylene (PTFE)) which may bond to the metallic reinforcement structure 64, the wire 71, and to one another. The metallic reinforcement structure 64 may be constructed of a mesh of between 16 and 32 of similar or varying material cords woven together (e.g., stainless steel, polyethylene naphthalate (PEN), polyethylene terephthalate (PET), and/or insulated electrical wire). The wire 71 is a solid core magnetic wire with a thin dielectric coating (e.g., a copper wire with a polyimide coating). One or more jackets formed of a polymeric material of the types described above may be employed to cover the metallic reinforcement structure 64. In embodiments where rather than a braid a laser cut tube is employed the jacket may be loosely formed to prevent its' becoming embedded in the cut portions of the laser cut tube to ensure that the laser cut tube can flex as described herein.

As depicted in FIGS. 3 and 4 the sensor 58 generally includes wrapping the wire 71 over the metallic reinforcement structure 64 of the catheter body 50 to form two wrapping layers 72 a, 72 b (FIG. 3) over the distal end portion 54 of the catheter body 50, and then is formed into a twisted pair 80 which is wrapped around the catheter body 50.

The wrapping layers 72a and 72b typically require a relatively high number of turns to obtain sufficient signal gain (e.g., about 200 turns of wire) to enable the sensor 58 to generate sufficient signal (induced current) when placed in an electromagnetic field. In addition, as a practical matter, when formed of a single wire 71, in order to allow for some human interaction with the wire 71 without breakage, even if just in loading the wire 71 into the machinery for forming the sensor 58, the wire must be up-sized wire diameter. Further, due to minimizing the wire size and the manufacturing process in general requires significant training and specialized equipment.

Further, use of a single wire places design constraint on the manufacture of the sensor 58. These constraints including wire diameter, wire strength, device wall thickness, and number of required turns results in a sensor of relatively fixed length, thickness, and gain all of which limit design flexibility.

FIG. 5 depicts a sensor 58 manufactured in a way that addresses and minimizes these constraints. The sensor 58 in FIG. 5 is formed of a continuous wire 71, however, rather than wrapping the wire 71 around the metallic reinforcement structure 64 as depicted in FIG. 3, the wire 71 is wrapped around a core 100. The core 100 is formed of a ferrous or electromagnetic field responsive material. The materials include stainless steel, steel, iron, cobalt, nickel, MP35N, alloys of each of these including mu metal. The core 100 may also be formed of ferrite powders, metal injection molding techniques, and sintering of feedstock materials. The core 100 may also be formed of a metallic glass, a metal powder-filled polymer. Still further the core 100 may formed of a liner which is spray or powder coated with materials having high magnetic susceptibility. The liner may alternatively be electrochemically plated with nickel, iron, cobalt or other ferromagnetic metals. All of the materials described above are ferromagnetic or at least induce an improved gain when placed in an electromagnetic field and may be selected based on their magnetic susceptibility to achieve the desired signal gain.

Regardless of which of the materials selected for the construction of the core 100a wire 71 is wrapped around the core 100. However, because of the ferromagnetic properties of the core, the wire 71 can be formed of a much thinner diameter than the embodiments of FIGS. 3 and 4. For example, the wire may between 56 and 42 gage (i.e., 0.000492 and 0.002494 inches in diameter). However, those of skill in the art will recognize that other sizes of wire may be employed without departing from the disclosure. The smaller diameter wire 71, when accompanied by a core 100 allows for a reduction in area of the sensor 58 or a reduction of the length of the sensor 58. In addition, the smaller diameter wire allows the increase in voltage to further improve signal gain and reduce noise. The wire 71 when wrapped to sufficient turns for formation of the electromagnetic sensor 58 have a much smaller cross-section than prior sensors 58. Further, by use of a core 100 that is ferromagnetic, the gain of the signal induced in the wires is actually increased as compared to sensors 58 composed of just the wrapped layers 72a and 72b of the wire.

As depicted in FIG. 5, the core 100 may also provide attachment points 102 for receiving pull wires 104. The pull wires 104 enable the steering of the distal end portion 54 of the catheter assembly 10. The pull wires 104 may be wires or pull threads without departing from the scope of the disclosure. This use of the core 100 for attachment of one or more pull wires 104 also reduces the size of the articulating means employed in current catheters.

The core 100 may also include a channel 106 that is molded, shaped, etched, or machined into the base 108 of the core 100. The channel 106 provides a path for the wire 71, which has been wound around the core 100 in two layers, to exit the core 100, but without increasing the overall diameter of the sensor 58 beyond the diameter of the core 100. As depicted in FIG. 5, immediately at the exit of the wire 71 from the core 100 the wire 71 may be formed into twisted pair 80 for wrapping around the catheter body 50 as described above. Still further, the core 100 may include a reduced diameter portion 51, the wrapping of the wire 71 in the reduced diameter portion 51 ensures that addition of the wire 71 to form the sensor 58 does not increase the diameter of the sensor 58 beyond the diameter of the core 100.

One benefit of the use of the core 100 is the increased resolution of the core 100 and therewith the distal portion 54 of the catheter assembly 10 when observed under fluoroscopy and other imaging techniques. Further the core 100 increases the resistance to crush of the sensor 58, and may be shaped to provide functionality. Thus, the core provides greater hoop rigidity. For example, the core may have a somewhat bullet shape to assist in navigating the catheter assembly 10 through for example airways, and after creation of an opening in the airways through such an opening.

The overcall dimensions of the core 100 may be sized to match that of the combination of the inner liner 60 and metallic reinforcement structure 64. In one embodiment, the sensor 58 including core 100 and wire 71 formed in wrapped layers 72a and 72b is formed separate from the inner liner 60 and metallic reinforcement structure 64. A proximal portion of the core 100 may be joined to the inner liner 60 and metallic reinforcement structure 64, for example by gluing or simply placed over a mandrel extending through the inner liner 60 and metallic reinforcement structure 64. The twisted pair 80 may also be fully formed separate from the placement of the core 100 proximate the inner liner 60 and metallic reinforcement structure 64. Because the twisted pair 80 has been formed as a twisted pair, the combination is much more stable than wire 71 alone and may be wrapped around the inner liner 60 and metallic reinforcement structure 64. Alternatively, a twisted pair 80 may be separately formed on the inner liner 60 and metallic reinforcement structure 64 and soldered to two ends of the wire 71 forming the wrapped layers 72 and 72b. Still further, the wire 71 may be supplied with the core 100 in bobbins. The wrapped layers 72a and 72b already formed on the core 100, and upon connection of the core 100 to the inner liner 60 and the metallic reinforcement structure 64 (e.g., placement on a common mandrel), the two bobbins may be placed in a wrapping machine which simultaneously forms the twisted pair 80 and wraps the twisted pair 80 around the inner liner 60 and the metallic reinforcement structure 64.

Once so formed with the core 100 with wrapped layers 72a and 72b formed thereon and a twisted pair extending and wrapped around the inner liner 60 and metallic reinforcement structure 64, and outer layer 68 may be slid onto the mandrel to cover the entire assembly. A reflow procedure heats the inner layer 60 and outer layer 68 causing the two layers to melt and form a single continuous layer of material with the braid entrapped therein. In the event that pull wires 104 are employed, their presence during the reflow procedure generally results in the formation of pull-wire channels allowing the movement of the pull wires 104 therein. Additionally or alternatively, a sleeve, as described in greater detail below may be employed to ensure freedom of motion of the of the pull-wire 104 after reflow of the inner liner 60 and outer liner 68. The sleeve could also be simply a groove formed in the outer liner 68.

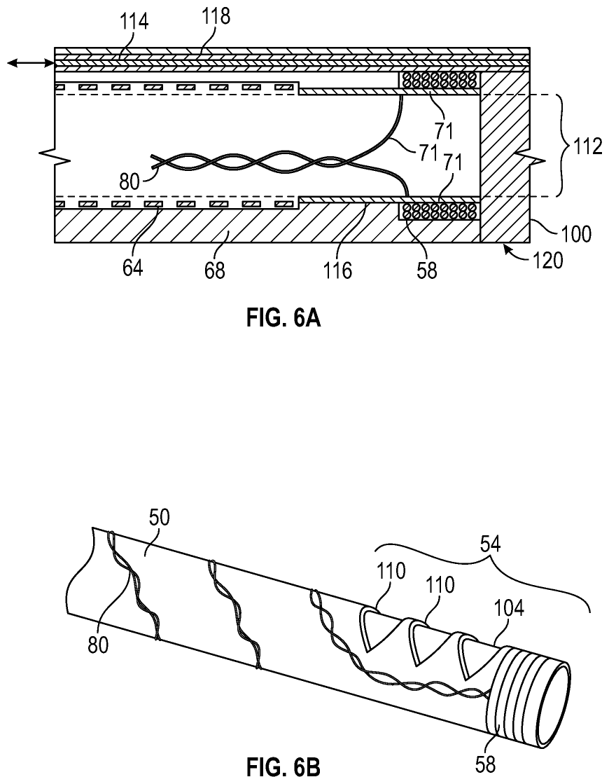

FIGS. 6A and 6B depict a further embodiment of the disclosure. FIG. 6A is a perspective view of a distal portion 54 of a catheter assembly 10. The distal portion 54 of the catheter assembly 10 includes a twisted pair 80 which is wrapped around the catheter body 50. The twisted pair 80 extends to the sensor 58. A pull wire 104 extends along the length of the catheter body 50 and terminates proximate the distal. As shown in FIG. 6A a series of fenestrations 110 may be cut into the catheter body 50 to allow for articulation of the distal portion 54 of the catheter body 50 upon retraction of the pull wire 104.

FIG. 6B depicts a cross-sectional profile view of a distal portion 54 of the catheter assembly 10 in accordance with the disclosure. Wire 71 is wrapped around a core 100. The core 100 has a lumen 112 formed therein. The diameter of lumen 112 substantially conforms to the diameter of the working channel 56 of the catheter assembly 10. As shown, the core 100 has an end portion 114 with an outer diameter substantially matching the outer diameter of the catheter assembly 10 (e.g., the outer liner 68). A metallic reinforcement structure 64 is formed in the catheter assembly 10, as described above, for enhanced rigidity while retaining desired flexibility of the catheter assembly 10. An inner liner 60 may be employed, as described above, but is not necessarily required. The metallic reinforcement structure 64 may terminate proximal of the distal end of the catheter assembly 10. Extending proximally from the end portion 114 of the core 100 is a cylinder 116. The outer diameter of cylinder 116 may substantially match the diameter of the metallic reinforcement structure 64. The cylinder 116 has an outer diameter that can be received within outer liner 68.

The wire 71 is wrapped around the cylinder 116, as described above, to form the sensor 58 and a twisted pair 80. The twisted pair 80 may be aligned such that it extends proximally along a centerline of a curve in the catheter assembly 10 created with the pull wire 104 is retracted to articulate the catheter assembly 10. The pull wire 104 extends along the length of the catheter assembly 10 in a pull wire sleeve 118. The pull-wire sleeve 118 ensures that the pull wire 104 is free the be retracted, even after the outer liner 68 and/or the inner liner 60 undergo a reflow process. The pull-wire sleeve 118 extends past the sensor 58 formed of wraps of wire 71 and prevents the sensor 58 from deforming when the catheter assembly 10 is articulated. The distal end of the pull wire 104 is anchored in the core 100. An outer sheath 120 may be placed over the outer liner 68 and the end portion 114 of the core 100.

As is depicted in FIG. 6B, during manufacture, the core 100 may be manufactured separately, and the wire 71 wrapped thereon to form the sensor 58 as a separate component. As described above, the inner liner 60 (if used), or metallic reinforcement structure 64 may be placed on a mandrel (not shown). The core 100 with sensor 58 formed thereon may be placed on the mandrel to abut the metallic reinforcement structure 64. The core 100 also includes the pull wire 114 attached thereto. The pull wire 114 may be welded to the core or adhered with an epoxy or other adhesive or other means of attachment. The pull wire 114 may be inserted into a pull wire sleeve 118 or the pull wire 114 and pull wire sleeve 118 may be combined prior to attachment to the core 100. The pull wire 114 and pull wire sleeve 118 may be laid on the metallic reinforcement structure 64 and temporarily held in place. The twisted pair 80 may then be wrapped around the metallic reinforcement structure 64 and the pull wire sleeve 118. An outer liner 68 may be placed over the twisted pair 80, metallic reinforcement structure 64, and pull wire sleeve 118. The outer liner 68 is then reflowed (e.g., heated above its melting temperature) such that it flows through the weave of the metallic reinforcement structure 64 and into any voids in the assembly between the metallic reinforcement structure 64 and the cylinder 116 of the core (e.g., portions proximal of the sensor 58). A sheath 120 may then be added, either before or after the reflow process and shrunk to the final size of the catheter assembly. This sheath 120 may be a reflow, a shrink wrap, or other material to cover the exterior of the catheter assembly 10.

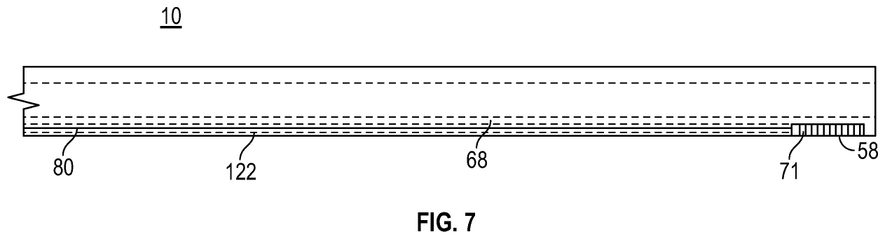

A further embodiment of the disclosure is seen in FIG. 7 where the core 100 occupies only a small portion of the distal end 54 of the catheter assembly 10. The core 100 is formed of one of the materials listed above and has a high ferromagnetic response. As a result, a smaller core 100 can be employed. The core 100 is wrapped with the wire 71, as described above, to form sensor 58. A twisted pair 80 extend from the core 100 along the length of the catheter assembly 10. The sensor 58, may be formed separately formed as a separate component and mated with the inner liner 60, metallic reinforcement structure 64, and outer liner 68 (all described above). Upon reflow of the inner and outer liners 60 and 68, or just outer liner 68 if no inner liner is employed, the reflow of the material effectively encapsulates the sensor 58 such that it is full encased in the reflow liner materials (e.g., the wall of the catheter assembly 10. The twisted pair 80 also becomes embedded in the liners during the reflow process. A lumen remains open through the extent of the catheter assembly 10 for the passage of tools, but there is no opening in the core 100.

In some embodiments either the inner liner 60 or the outer liner 68 may or a polymeric jacket as described herein may not be formed of a reflow material, but rather of a non-reflow polymeric material. Additionally, the inner liner 60, outer liner 68 or the polymeric jacket may be formed of sections of material some of which are reflow materials and some are non-reflow materials. In one aspect of the disclosure either non-reflow materials are employed or if reflow materials are employed the outer liner 68 or the polymeric jacket is not reflowed in the region proximal of the core 100 and throughout the area of the articulation, thus creating a loose fitting cover over those portions of the metallic reinforcement structure 64. In accordance with a further aspect of the disclosure the outer liner 68 or the polymeric jacket can have a variety of states along the length of the metallic reinforcement structure 64 of the catheter body 50. In some areas the outer liner 68 or the polymeric jacket may be highly reflowed to fuse with the inner liner 60. In other areas the outer liner 68 or the polymeric jacket may be minimally reflowed such that it infuses the metallic reinforcement structure 64 but does not fuse with the inner liner 60. Further the outer liner 68 or the polymeric jacket may be not reflowed at all along any portion of the catheter body 50.

In accordance with another aspect of the disclosure in FIG. 7, rather than wrapping the twisted pair around the outer liner 68, a second sleeve 122 similar to pull-wire sleeve 118 may be employed to receive the twisted pair 80. As with the pull wire sleeve 118, the sleeve 122 for the twisted pair 80 may be placed over the twisted pair, and then the sleeve 122 temporarily adhered or affixed to the outer liner prior to a reflow step to permanently affix the sleeve to the outer liner 68. Again, a groove (not shown) may be formed in the outer liner 68 to receive the twisted pair as an alternative to the sleeve 122. Though a pull wire sleeve 118 and second sleeve 122 (or grooves) are described herein, similar features and functionality can be achieved using a single sleeve in which both the twisted pair 80 and the pull reside.

Still further, rather than inserting the twisted pair 80 into a sleeve 122 or a groove formed in the outer liner 68, the twisted pair could be co-extruded with the outer liner 68. At a distal end of the outer liner 68, the twisted pair could exit the outer liner and be positioned to connect to ends of the wire 71 used to form sensor 58. The ends of the wire 71 and the ends of the twisted pair could be soldered together, using for example a solder pad connection. Alternatively the ends of the wire 71 and the twisted pair 80 could be laser welded together. Still further micro-crimping could be employed to physically and electrically connect the ends of the twisted pair 80 and the ends of the wire 71 such that the sensor 58 is in electrical communication with the twisted pair 80 along the length of the catheter assembly 10.

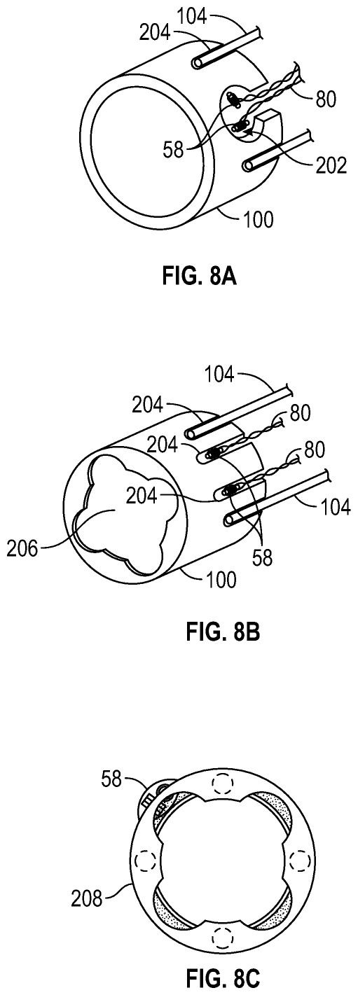

Additional aspects of the disclosure regarding the core 100 and particularly the attachment of the pull wires 104 and sensors 58 to the core 100. In FIG. 8A the core 100 includes a cut-out 202 into which are led a pair of sensors 58. Each sensor 58 is connected to via a twisted pair 80. The pull wires 104, extend into slots 204 formed in the core 100. Both the sensors 58 (and therewith the distal ends of the twists pairs 80) and the pull wires 104 can be mounted or potted in the core using for example epoxy or other potting or encapsulating materials to prevent the pull wires 104 from coming out of the core 100 during articulation of the catheter assembly 10. FIG. 8B depicts another variation of a core 100. Rather than a cut-out 202 each of the sensors 58 and their respective twisted pair 80 are inserted into a slot 204 similar to the slots 204 into which the pull wires 104 are inserted. As can be seen the distal face 206 of the core has clover leaf design formed therein. The clover leaf configuration is a tool retention cap 208 that may be integrally formed with the core 100 or can be a separate component removable from the core 100. For example, as seen in FIG. 8C, the tool retention cap 208 can be used to ensure that a camera or tool greater than a certain size is retained within the catheter assembly 10. In addition, in FIG. 8C, the end view of the catheter assembly 10 and specifically the tool retention cap 208 the placement of the sensors 58 may protrude from the circumference of the tool retention cap 208 as well as extend into an open portion of the core 100. In the embodiment of FIG. 8C, the open cross-section of the catheter assembly 10 around the camera or tool allows for the suction of or removal of fluids from the patient during navigation. Use of the cut-out 202 and slots 204 along with the metallic nature of the core 100 may provide the opportunity to eliminate the wrapping of a sensor wire 71 around a reduced diameter portion of the core 100.

It will be recognized that while certain aspects of the disclosure are described in terms of a specific sequence of steps of a method, these descriptions are only illustrative of the broader methods of the disclosure and may be modified as required by the application. Certain steps may be rendered unnecessary or optional under certain circumstances. Additionally, certain steps or functionality may be added to the disclosed embodiments, or the order of performance of two or more steps permuted. All such variations are encompassed within the disclosure.

While the above detailed description has shown, described, and pointed out novel features of the disclosure as applied to various embodiments, it will be understood that various omissions, substitutions, and changes in the form and details of the device or process illustrated may be made by those skilled in the art without departing from the disclosure. The foregoing description is of the best mode presently contemplated of carrying out the disclosure. This description is in no way meant to be limiting, but rather should be taken as illustrative of the general principles of the disclosure. The scope of the disclosure should be determined with reference to the claims.

We claim:

1. A catheter comprising:
an inner liner defining a first lumen;
an outer liner formed over the inner liner;
a metallic reinforcement structure between the inner liner and the outer liner;
a first sleeve operably connected to the outer liner;
a core in communication with the inner liner and having a second lumen of substantially the same diameter as the first lumen and a reduced diameter portion;
a pull wire traversing the first sleeve and anchored to the core; and
a sensor wire, having a first portion of the wire is formed in a plurality of windings wrapped around the reduced diameter portion of the core and a second portion of the wire formed as a twisted pair extending proximally along the outer liner; and
an outer sheath exterior to the outer liner, the first sleeve, and the core.

2. The catheter of claim 1, further comprising a second sleeve operably connected to the outer liner and within the outer sheath, wherein the twisted pair extends through the second sleeve.

3. The catheter of claim 1, further comprising a plurality of fenestrations formed in the outer liner and configured to allow the pull wire to articulate a distal portion of the catheter.

4. The catheter of claim 1, wherein twisted pair wraps around the outer liner and is captured by the outer liner.

5. The catheter of claim 1, wherein the core includes a channel for passage of the twisted pair.

6. The catheter of claim 1, wherein the core includes a cylinder formed on a proximal portion, the cylinder having an inner diameter substantially the same as the lumen in the inner liner.

7. The catheter of claim 6, wherein the plurality of windings are formed on the cylinder.

8. A catheter assembly comprising:
a catheter body formed of one or more polymeric materials, the catheter body defining a first lumen therethrough;
a metallic reinforcement structure in contact with the one or more polymeric materials;
a core distal of the metallic reinforcement structure and the one or more polymeric materials; and
a wire, wherein a first portion of the wire is formed in a plurality of windings wrapped around the core and a second portion of the wire formed as a twisted pair extending proximally along the catheter body.

9. The catheter assembly of claim 8, wherein the core includes a second lumen formed therein substantially the same diameter as the first lumen.

10. The catheter assembly of claim 8, wherein the core includes a channel for passage of the twisted pair.

11. The catheter assembly of claim 8, wherein the core includes a reduced diameter portion for receiving the wire and forming the plurality of windings.

12. The catheter assembly of claim 8 further comprising a sleeve formed in the catheter body.

13. The catheter assembly of claim 12, further comprising a pull wire traversing the sleeve and attached to the core, wherein actuation of the pull wire articulates a distal portion of the catheter assembly.

14. The catheter assembly of claim 13, further comprising a plurality of fenestrations formed in the catheter body and configured to allow the pull wire to articulate a distal portion of the catheter.

15. The catheter of claim 8, wherein the catheter body includes an outer liner and the twisted pair is captured by the outer liner.

16. The catheter of claim 8, wherein the core includes a channel formed for passage of the twisted pair.

17. The catheter of claim 8, wherein the catheter body includes an inner liner and the core includes a cylinder formed on a proximal portion, the cylinder having an inner diameter substantially the same as a diameter of a lumen in the inner liner.

18. The catheter of claim 17, wherein the plurality of windings are formed on the cylinder.

19. A catheter assembly comprising:
a catheter body formed of one or more polymeric materials, the catheter body defining a first lumen;
a metallic reinforcement structure in contact with the one or more polymeric materials;
a core distal of the metallic reinforcement structure and embedded in a wall of the catheter body, the core covered by at least one of the one or more polymeric materials, the core extending partially around a diameter of the catheter body; and
a sensor wire, wherein a first portion of the wire is formed in a plurality of windings wrapped around the core and a second portion of the wire formed as a twisted pair extending proximally along the catheter body.

20. The catheter assembly of claim 19, further comprising a sleeve operably connected to the catheter body and embedded in the one or more polymeric materials, wherein the twisted pair traverses the sleeve.
